(19) United States
(12) Patent Application Publication
(10) Pub. No.: US 2014/0200460 A1
(43) Pub. Date: Jul. 17, 2014

(54) REAL-TIME PHYSIOLOGICAL CHARACTERISTIC DETECTION BASED ON REFLECTED COMPONENTS OF LIGHT

(71) Applicants: Matthew Johnson, Hillsborough, CA (US); Aza Raskin, San Francisco, CA (US)

(72) Inventors: Matthew Johnson, Hillsborough, CA (US); Aza Raskin, San Francisco, CA (US)

(73) Assignee: AliphCom, San Francisco, CA (US)

(21) Appl. No.: 13/967,317

(22) Filed: Aug. 14, 2013

Related U.S. Application Data
(60) Provisional application No. 61/682,854, filed on Aug. 14, 2012.

(51) Int. Cl. A61B 5/00; A61B 5/024

(52) U.S. Cl. CPC A61B 5/0077; A61B 5/024
USPC 600/479; 600/476

(57) ABSTRACT

Embodiments relate generally to electrical and electronic hardware, computer software, wired and wireless network communications, and wearable/mobile computing devices configured to facilitate health and wellness monitoring and maintenance. More specifically, disclosed are systems, components and methods to detect physiological characteristics, such as heart rate, of an organism in real-time based on components of light. In various embodiments, a method can include receiving color channel signals including imagery data generated by, for example, an image capture device. A linear combination of the color channel signals can form a combined color channel signal. The method also can include transforming continuously the combined color channel signal to establish local maxima associated with multiple scales. Further, portions of time associated with the local maxima can be identified and data signal representing a physiological characteristic can be generated. A local maximum can indicate the presence of enhanced blood volume adjacent a tissue surface.

### CROSS-REFERENCE TO RELATED APPLICATIONS

This U.S. non-provisional patent application claims the benefit of, and priority to, U.S. Provisional Patent Application No. 61/682,854 filed on Aug. 14, 2012, which is incorporated by reference herein for all purposes.

### FIELD

Embodiments relate generally to electrical and electronic hardware, computer software, wired and wireless network communications, and wearable/mobile computing devices configured to facilitate health and wellness monitoring, maintenance, and the like. More specifically, disclosed are systems, components, and methods to detect physiological characteristics, such as heart rate, of an organism in real-time based on components of light.

### BACKGROUND

Physiological characteristics of an individual, such as heart rate, can be monitored and measured to determine various health and wellness aspects of the individual, such as health, fitness, interests, activity level, awareness, mood, engagement in an activity, etc. Various techniques and approaches of measuring heart rate currently exist, from finding a pulse and counting beats over a period of time to using an EKG machine. However, each of these methods requires contact with the individual, and the former providing a significant distraction to the individual and the latter requiring expensive equipment.

Recent developments in health and wellness include the recovery of information regarding facial flushing using video data from a camera, such as a webcam. Facial flushing (or flushes) can indicate the presence of blood, at or near the surface of tissue, due to activity/exercise or changes in mood (e.g., angry, embarrassed, etc.). Conventional approaches to determining heart-related data from video data, while functional, are not well-suited for relatively immediate physiological characteristic extraction. For example, typical approaches to deriving heart rates from video data rely on “batch processing” and/or “complex pre- and post-processing.” Such approaches usually require that RGB pixel data are to be averaged over the spatial dimensions of, for example, a user's face, whereby the averaging is performed independently each time for each unit of area. Further, complex processing steps are typically required, and are traditionally batched. For example, a “detrending” operation to move/remove low-frequency components and/or a “normalization” operation are performed in “batches,” or in relatively large non-continuous groups of data. Further, signal separation algorithms and/or filter processing (e.g., via a 128-tap bandpass finite impulse response filter) are also usually performed in batches, too. Such filters also typically generate numerous parameters requiring computing resources. Then, peaks in resulting waveforms of the batched signals are used to estimate heart beats. The aforementioned operations and requirements typically require relatively large amounts of time and/or computational resources than otherwise might be the case due to, for example, the batched processing, which results in non-continuous processing.

Thus, there is a need to create novel methods for detecting physiological characteristics, such as heart rate, of an organism.

### BRIEF DESCRIPTION OF THE DRAWINGS

Various embodiments or examples (“examples”) of the invention are disclosed in the following detailed description and the accompanying drawings:

FIG. 1 is a functional block diagram depicting an implementation of a physiological characteristic determinator, according to some embodiments;

FIGS. 2 to 3B depict various examples of implementing a physiological characteristic determinator, according to various embodiments;

FIG. 4 depicts a flow for determining a physiological characteristic, according to some embodiments;

FIG. 5 is a diagram depicting a real-time physiological signal extractor and examples of its components, according to some embodiments;

FIG. 6 depicts a flow for estimating heart rate, according to some embodiments;

FIG. 7 depicts an example of a color signal combiner, according to some embodiments;

FIGS. 8A and 8B depict an example of a wavelet transformer, according to some embodiments;

FIG. 9 depicts examples of a maxima detector and a multi-scale physiological estimator, according to some embodiments;

FIG. 10 depicts an example of a physiological signal generator, according to some embodiments;

FIG. 11 depicts an example of a set of tunable parameters, according to some embodiments; and

FIG. 12 illustrates an exemplary computing platform disposed in a media device, a mobile device, a wearable device, or any computing device, according to various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

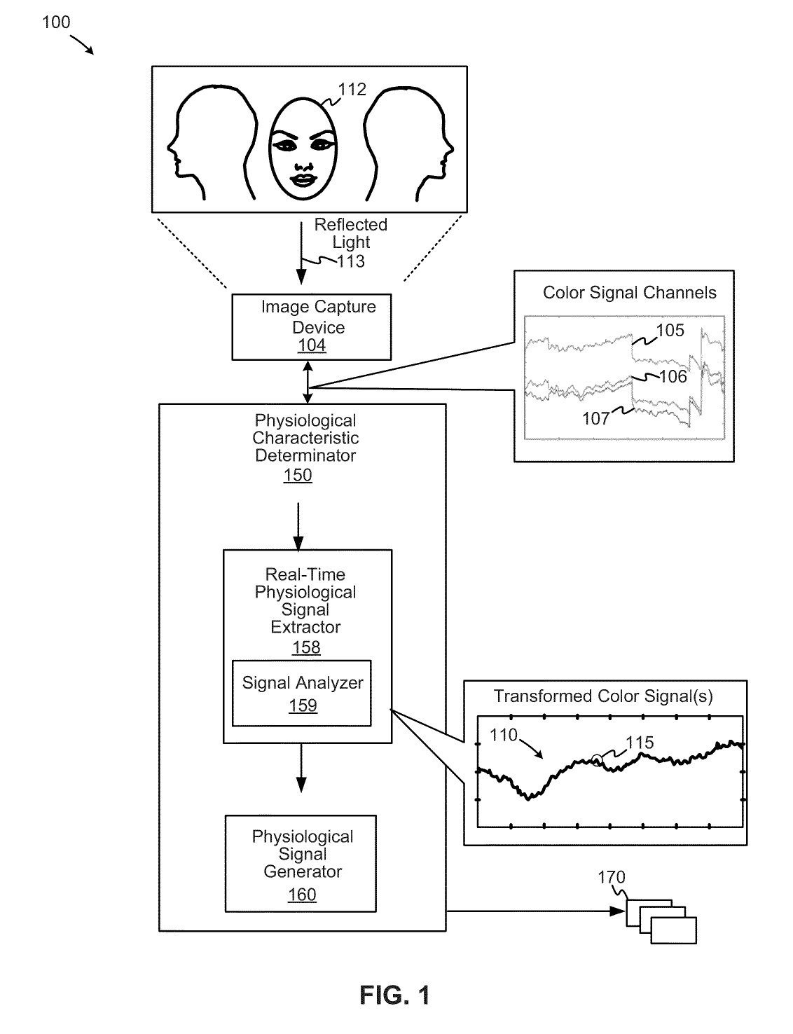

FIG. 1 is a functional block diagram depicting an implementation of a physiological characteristic determinator, according to some embodiments. Diagram 100 depicts a physiological characteristic determinator 150 that is coupled to an image capture device 104, which can be a digital camera (e.g., video camera). As shown, physiological characteristic determinator 150 includes a real-time physiological signal extractor 158 and a physiological signal generator 160.

Real-time physiological signal extractor 158 is configured to extract one or more signals including physiological information from a real-time stream of data representing subsets of light components captured as reflected light 113 by image capture device 104. For example, each subset of light components of reflected light 113 can be associated with one or more frequencies. Reflected light 113 can originate from tissue, such as a face 112 of an organism or person, whereby reflected light 113 can include physiological information based on face flushing or flushes (e.g., can include plethysmographic signal information). A face flushing state can be accompanied by an enhanced blood volume, at least in some cases, at or near the surface of face 112 tissue, whereby a non-flushed state indicates a relatively lower blood volume. According to some embodiments, the transitions between face flushing states and non-flushing states can be due to pulsations of blood (i.e., heart beats), from which heart-related information (e.g., heart beat timings, heart rate, etc.) may be acquired via reflected light 113 (or other electromagnetic waveforms).

According to some embodiments, real-time physiological signal extractor 158 identifies a first subset of frequencies (e.g., a range of frequencies, including a single frequency) constituting green visible light, a second subset of frequencies constituting red visible light, and a third subset of frequencies constituting blue visible light. Other frequencies and wavelengths are possible, including those outside the visible spectrum. As shown, physiological characteristic determinator 150 receives red visible light via a red color channel 105, green visible light via a green color channel 106, and blue visible light via a blue color channel 107, any of which can include pixel values or other color-related signal values.

According to some embodiments, real-time physiological signal extractor 158 is configured to implement wavelet transforms, such as a continuous wavelet transform (“CWT”), to detect heart beats and to estimate a heart rate from video in real-time, rather than, for example, processing light component data in batches. According to some examples, a batch step, batch process, or batch algorithm can refer to a function or operation that is not well-adapted to implementation as a real-time application and/or does not operate sufficiently on continuous signals and wavelet transforms. Such batch steps, processes, or algorithms may be described, in some instances, in terms of a batch processing pipeline through which data processing is non-continuous and/or not in real-time (or not substantially in real time), such as static data (or transitory static data). In some examples, a batch step, process, or algorithm may include iterations (e.g., to maximize data, minimize data, diagonalize data, etc.), and, as such, may process static data or temporally stored data (e.g., data other than that in a real-time data stream). Thus, batch steps, processes, or algorithms may not be configured to operate upon real-time data streams.

Real-time physiological signal extractor 158 is configured to apply continuous wavelet transforms to data representing color-related signal values of, for example, a linearly-combined color channel signal to form a transformed color signal 110. The continuous wavelet transform is configured to yield local maxima at different scales or values of scale parameters. When a local maximum is present over a threshold number of adjacent or related scales (e.g., over a number of continuous values of scales), real-time physiological signal extractor 158 can specify that the local maximum is a “ridge.” According to various embodiments, the presence of a local maximum and/or ridge indicates a flushing state (e.g., the presence of sufficient blood volume at or near the surface of the face tissue).

According to some examples, signal analyzer 159 of real-time physiological signal extractor 158 can identify a time-domain component associated with a change in blood volume associated with the one or more surfaces of the organism. Thus, signal analyzer 159 is configured to analyze the local maxima and/or ridges associated with a combined color channel signal to identify points in time or portions of time during which flush pulses likely coincide. For example, signal analyzer 159 can specify an interval of time 115 that may indicate a flushing state with which timing can be determined relative to other flushing state pulses and/or ridges (not shown). The timing data specifying multiple intervals of time can be provided to physiological signal generator 160.

Physiological data signal generator 160 can be configured to generate a physiological data signal 170 representing one or more physiological characteristics. Examples of such physiological characteristics include heart beats per unit time, a heart rate, a pulse wave rate, a heart rate variability (“HRV”), and a respiration rate, among others, in a non-invasive manner. In at least one example, the timing information (e.g., the frequency or period) of sequential occurrences of flushing state pulses, ridges, or local maxima can be used to determine heart beat per unit time, or a heart rate.

In view of the foregoing, the functions and/or structures of one or more of physiological characteristic determinator 150, real-time physiological signal extractor 158, and physiological signal generator 160 can provide for the determination of wavelet-based estimation of physiological characteristics, such as a heart rate. Physiological characteristic determinator 150 can operate to apply continuous wavelet transforms on real-time data streams. Further, identification of the local maxima and/or ridges need not require relatively computationally-intensive pre-processing, such as detrending and/or centering or normalization. In some cases, physiological characteristic determinator 150 need not require relatively computationally-intensive post-processing, such as cubic spline interpretation or convolving operations against a linear time-invariant (“LTI”) band-pass filter. Additionally, real-time physiological signal extractor 158 can operate to detect physiological characteristics, such as heart beats, in the time domain. Thus, real-time physiological signal extractor 158 need not require frequency domain operations, such as a Short-Time Fournier Transform (“STFT”), which may require computations associated with the Gabor limit or the Heisenberg Uncertainty Principle to reduce or negate, for example, blurring effects of STFT representations. In some cases, the use of a continuous wavelet can address frequency variation of a color signal over time. Moreover, physiological characteristic determinator 150 can forgo use of batch processing, which may consume more computational resources and/or time than otherwise might be the case. Note that the above-described indications that real-time physiological signal extractor 158 (and its components) do not require certain structures or functions is not intended to exclude such structures and functions from the scope of the disclosure herein or the claimed invention(s). Also, the detections of a local maximum over multiple (e.g., several) contiguous scales can facilitate reduction or negation of the effects of noise and/or variations of in the underlying heart rate, among other things.

In some embodiments, physiological characteristic determinator 150, and a device in which it is disposed, can be in communication (e.g., wired or wirelessly) with a mobile device, such as a mobile phone or computing device. In some cases, such a mobile device, or any networked computing device (not shown) in communication with physiological characteristic determinator 150, can provide at least some of the structures and/or functions of any of the features described herein. As depicted in FIG. 1 and subsequent figures (or any figures herein), the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1 (or any figure) can represent one or more algorithms. Or, at least one of the elements can represent a portion of logic including a portion of hardware configured to provide constituent structures and/or functionalities.

For example, physiological characteristic determinator 150 and any of its one or more components, such as real-time physiological signal extractor 158 and physiological signal generator 160, can be implemented in one or more computing devices (i.e., any video-producing device, such as mobile phone, a wearable computing device, such as UP® or a variant thereof), or any other mobile computing device, such as a wearable device or mobile phone (whether worn or carried), that include one or more processors configured to execute one or more algorithms in memory. Thus, at least some of the elements in FIG. 1 (or any figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques (as well as other structures and techniques described herein) can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, physiological characteristic determinator 150 and any of its one or more components, such as real-time physiological signal extractor 158 and physiological signal generator 160 can be implemented in one or more circuits. Thus, at least one of the elements in FIG. 1 (or any figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

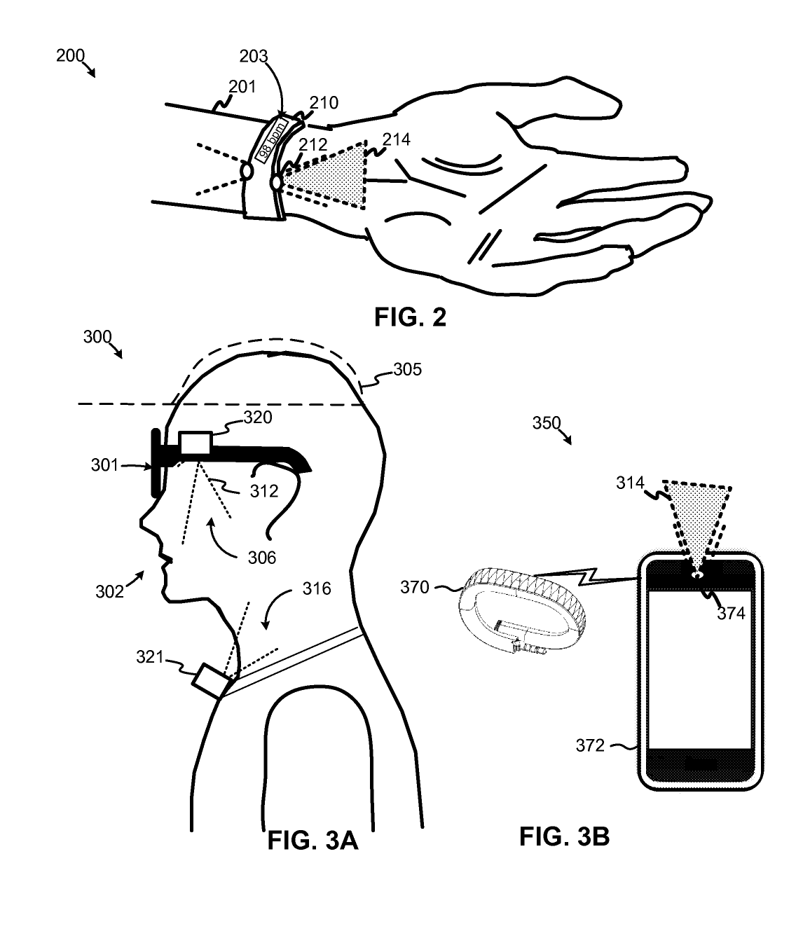

FIG. 2 depicts a wearable device 210 implementing a physiological characteristic determinator, according to some embodiments. The physiological characteristic determinator (not shown) is coupled to one or more light capture devices 212 (e.g., image capture devices) to receive reflected light from surface portions 214. As shown, wearable device 210 is dispose on an organism's wrist and/or forearm, but can be located anywhere on a person. Wearable device 210 can include a display 203 (e.g., LED, LCD, capacitive, touch-sensitive, etc.) to present, for example, heart rate information. An example of a suitable wearable device, or a variant thereof, is described in U.S. patent application Ser. No. 13/454,040, which was filed on Apr. 23, 2012, which is incorporated herein by reference.

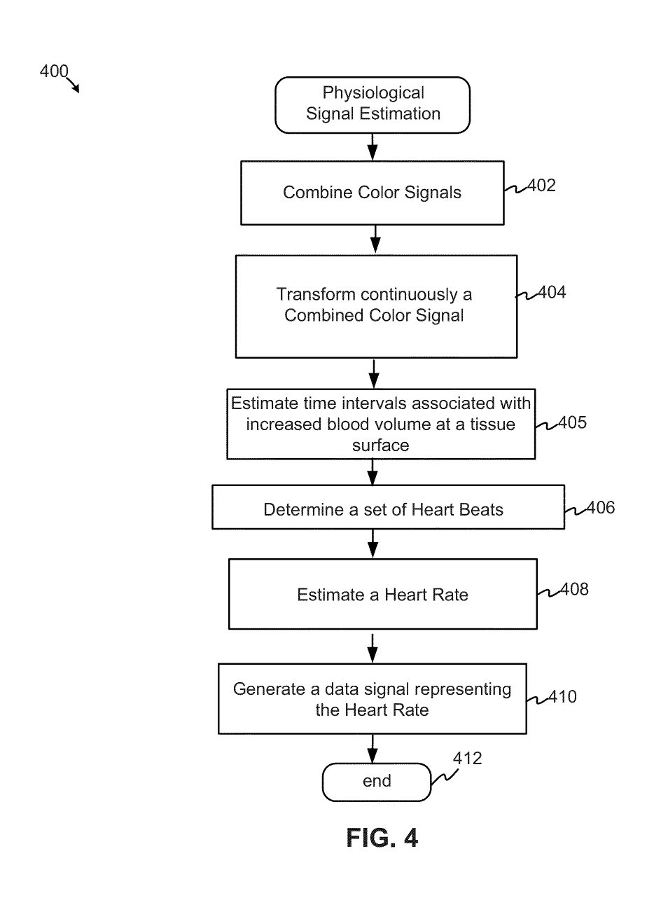

FIG. 3A depicts a wearable article or portion of apparel implementing a physiological characteristic determinator, according to some embodiments. Diagram 300 depicts physiological characteristic determinator disposed in a housing 320, which can couple to eyewear 301, a hat 305, or clothing of organism 302. The physiological characteristic determinator in housing 321 is coupled to clothing, such as a shirt collar, to receive light reflected by area 316, under which are relatively detectable volumes of blood fluctuate and change over time. The physiological characteristic determinator in housing 320 is coupled to eyewear 301 to receive light 312 reflected by area 306, which is one of a number of face portions from which light is reflected.

FIG. 3B depicts a mobile computing device configured to implement a physiological characteristic determinator, according to some embodiments. Diagram 350 depicts one or more portions of physiological characteristic determinator being disposed in housing 370 or housing 372, or a combination thereof. Distributed portions of the physiological characteristic determinator can communicate via a wireless link. The physiological characteristic determinator (not shown) is coupled to one or more light capture devices 374 to receive reflected light from surface portions 314 (e.g., a portion of tissue adjacent housing 372 during a telephone call or at a distance when, for instance, exercising). Physiological characteristic determinator can be disposed in any electronic device, such as a media device, examples of which are disclosed in U.S. patent application Ser. No. 13/831,422, entitled “Proximity-Based Control of Media Devices,” filed on Mar. 14, 2013 with Attorney Docket No. ALI-229, which is incorporated herein by reference. In various examples, media device 140 is not limited to presenting audio, but rather can present and/or receive both visual information, including video (e.g., using a pico-projector digital video camera and/or projector) or other forms of imagery, optionally along with (e.g., synchronized with) audio. At least some components of the media device can be implemented similarly as Jambox® products produced by AliphCom of California.

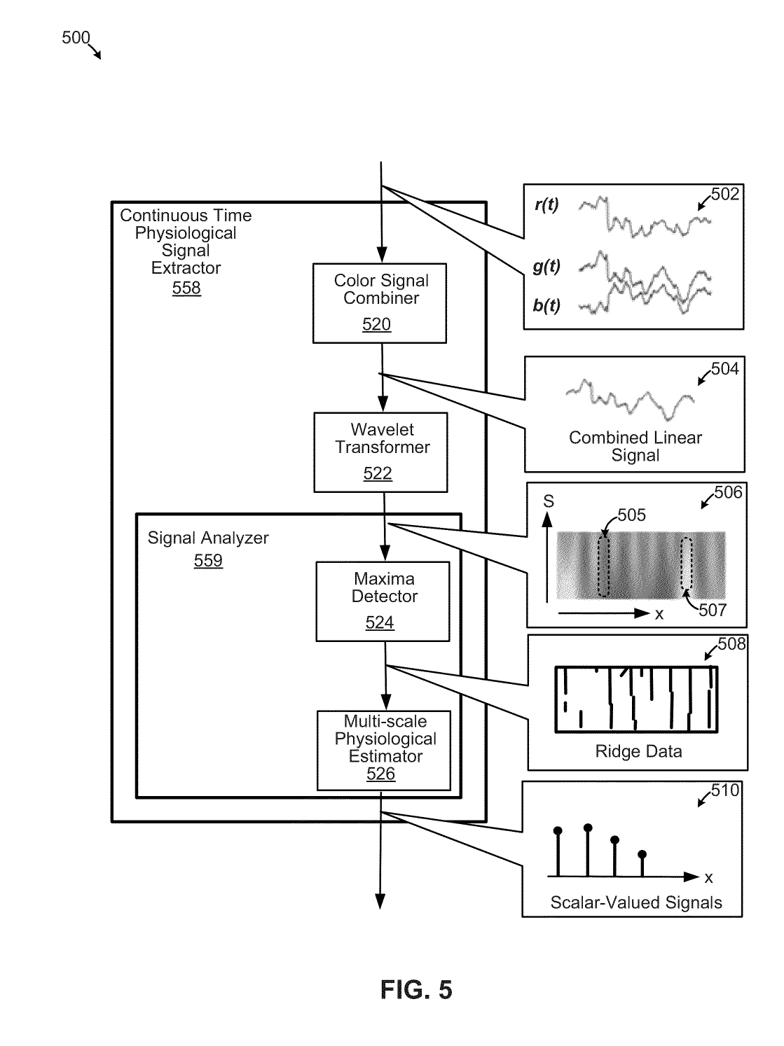

FIG. 4 depicts a flow for determining a physiological characteristic, according to some embodiments. Flow 400 provides for the estimation of a physiological characteristic, such as the heart rate (“HR”) of a subject or organism. As shown, flow 400 begins at 402, whereby a combination (e.g., a linear combination) of color channel signals is formed as a combined color channel signal. The color channel signals include data representing one or more images received from an image capture device. Note that the color channel signals can be RGB, CMYK, or any other color space. At 404, the combined color signal is transformed continuously (or substantially continuously), for example, at a processor to establish to identify portions of a transformed combined color signal that indicates durations of time associated with the combined color channel signal in which tissue is in a flushed state (e.g., an enhanced amount of blood is at or near the surface). Based on the identified portions generated by a transform operation that are associated with increased blood volume, estimated time intervals (e.g., the timing) between the identified portions are determined at 405. At 406, a set of physiological characteristics, such as heartbeats, is determined, and a heart rate is estimated therefrom at 408. At 410, data representing a physiological signal is generated, the physiological signal including, for example, the heart rate. Flow 400 terminates at 412.

In the foregoing or any other variation, the video signal and pixel data and values of the color signal channels are preferably a live feed from the camera in the electronic device, though the video signal can be preexisting, such as a video signal recorded previously with the camera. Alternatively, a video signal including the color signal channels can be sent to the electronic device, or downloaded from a remote server, network, or website. Furthermore, the flow 400 need not be limited to heart rate determination, and can also include, or be directed to, calculating the heart rate variability (“HRV”) of the subject and/or calculating the respiratory rate (“RR”) of the subject, or any other physiological characteristic, such as a pulse wave rate, a Meyer wave, etc.

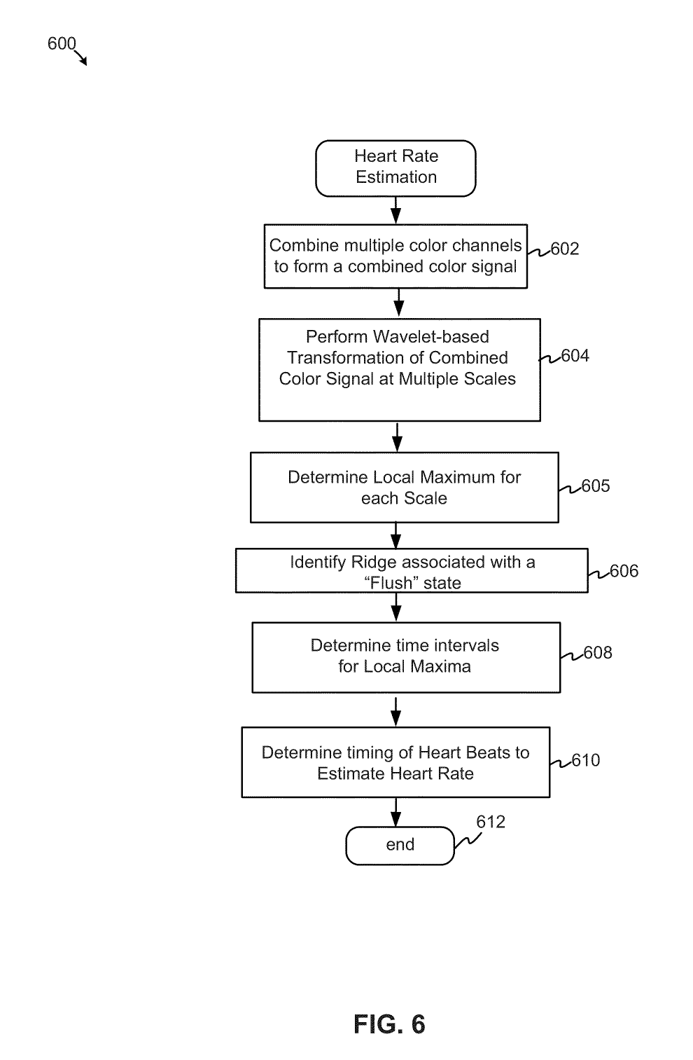

FIG. 5 is a diagram depicting a real-time physiological signal extractor and examples of its components, according to some embodiments. Diagram 500 depicts a real-time physiological signal extractor 558 including a signal analyzer 559. As shown, real-time physiological signal extractor 558 includes a color signal combiner 520 and a wavelet transformer 522. Further, signal analyzer 559 is shown to include a maxima detector 524 and a multi-scale physiological estimator 526.

Color signal combiner 520 is configured to form a linear combination of multiple color channels 502, such as one or more of red color channel, r(t), green color channel, g(t), and blue color channel, b(t). Based on multiple color channel signals 502, color signal combiner 520 generates a combined linear color signal 504. Wavelet transformer 522 is configured to receive combined linear signal 504, and is further configured to transform continuously signal 504 over multiple scales (e.g., a couple of, or several scales) to generate data represented by a plot 506, which depicts local maxima 507 with light shading and local minima 505 with dark shading. The Y-axis represents the scale (“s”), which can include values of scale parameters for a continuous wavelet transform implemented by wavelet transformer 522, whereas the X-axis represents parameter, x, which, in turn, can represent time, t, a number of samples, n, or the like. According to various embodiments, wavelet transformer 522 can be configured to perform any of one or more continuous wavelet transforms.

Maxima detector 524 is configured to identify local maxima, and is further configured to determine whether each of the local maxima constitute a wavelet transform “ridge.” As local maximum 507 is depicted as extending contiguously over multiple scales (i.e., the light shading extends over multiple values of scale), local maximum 507 is tagged as a “ridge.” Maxima detector 524 also generates a set of ridge data 508 that specifies a location of various ridges and specific intervals of time. Multi-scale physiological estimator 526 is configured to operate over the multiple scales of wavelet transform to generate scaler-valued signals 510 representing the timing at which flush states are detected. The timing then can be used to identify heart beats along the X-axis (e.g., in units of time). Note that in some optional examples, the amplitude of scaler-valued signals 510 can represent a relative amount of blood associated with a portion of tissue (e.g., a cheek on the face of a user).

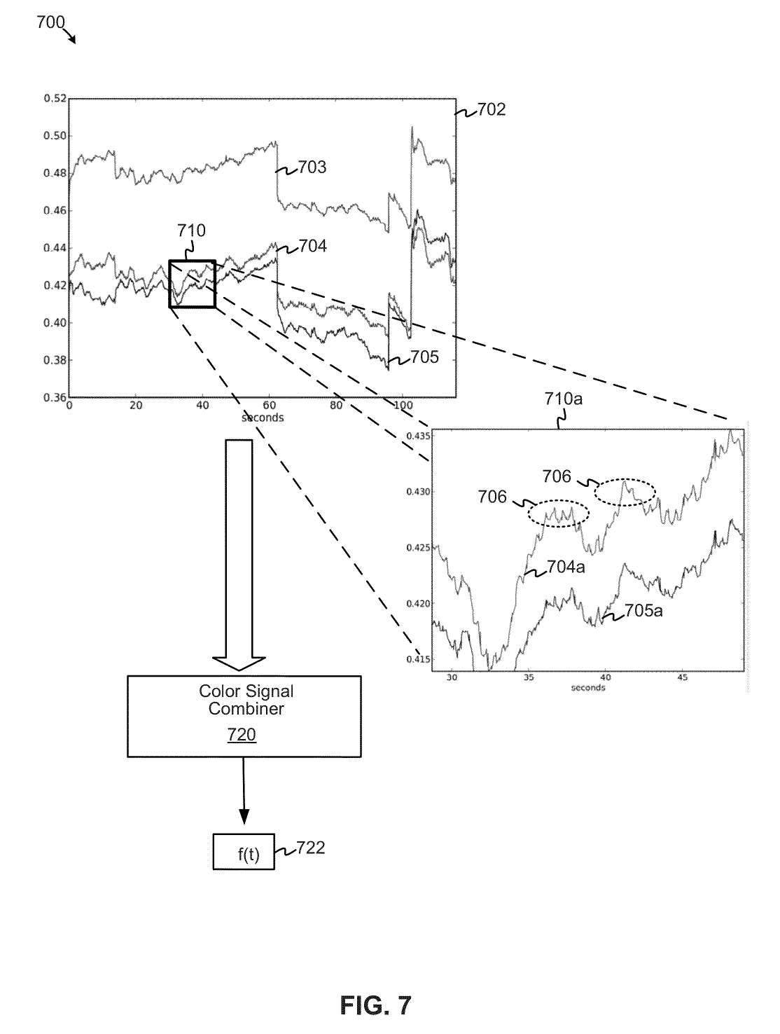

FIG. 6 depicts a flow for estimating heart rate, according to some embodiments. Flow 600 provides for the estimation of heart rate (“HR”) of a subject or organism. As shown, flow 600 begins at 602, whereby a combination of color channel signals is formed as a combined color channel signal. In at least one embodiment, the contribution of the green color channel predominates over the other changes (i.e., the red and blue color channels). In one example, the combined color channel signal is formed based only on the green color channel. At 604, a wavelet-based transformation of the combined color signal is performed at multiple scales in real time or substantially in real-time to form a transformed combined color channel signal (as a substantially continuous time signal). Further, the combined color channel signal is transformed continuously, for example, at a processor to establish local maxima associated with multiple scale parameters (e.g., values of scale parameters) at 605. In some embodiments, while combined color channel signal may undergo a delay in a transformation of the same, so long as the operations are continuous, it can be view as being in substantially real-time.

In some embodiments, a local maximum can establish a ridge when, for example, the local maximum resides substantially at the same time point over contiguous, adjacent scales (e.g., values of scale parameters). At 605, portions of time associated with the local maxima are identified to estimate time intervals associated with increased blood volume in a tissue surface. Ridges associated with a “flush” state can be identified at 606, whereby the determination of a ridge, for example, can be based on the detection of a local maximum that spans over a threshold number of contiguous scales in a duration of time. At 608, time intervals associated with the local maxima are identified to estimate timing between associated increases in blood volume in a tissue surface (i.e., sequential flush states). At 610, the timing of heart beats associated with ridges is used to estimate the heart rate. Flow 600 terminates at 612.

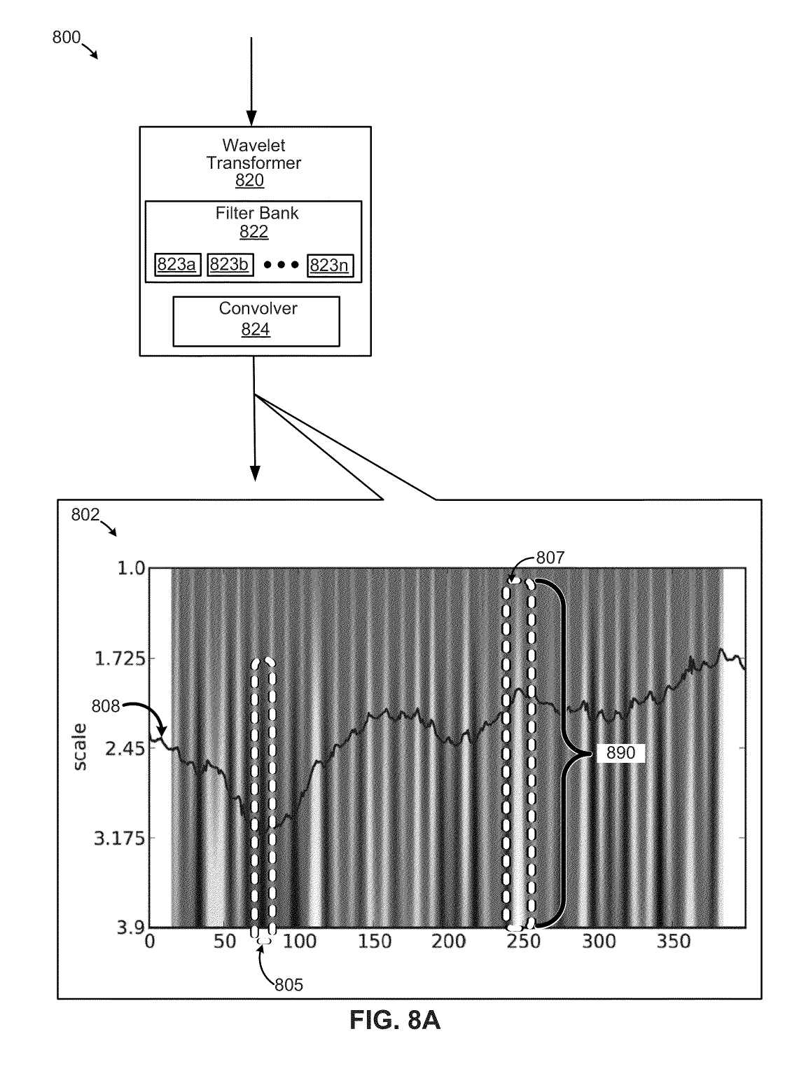

FIG. 7 depicts an example of a color signal combiner, according to some embodiments. Diagram 700 depicts a color signal combiner 720 being configured to receive multiple color channel signals 702, which, as shown, includes a red channel signal (“r(t)”) 703, the green channel signal (“g(t)”) 704, and a blue channel signal (“b(t)”) 705. Diagram portion 710a represents an enlarged version of 710, whereby peak values 706 are can be more readily identifiable in relation to enlarged green color channel signal 704a and enlarged blue color channel signal 705a. Peak values 706 can arise due to data received indicating states of flush. The flushes of interest (or data representations thereof) can be pulses that are shown to occur, for example, approximately 0.5 to 1.0 seconds apart. In some example, peak values 706 may be more evident in the green channel. Color signal combiner 720 generates a “flushing signal” (“f(t)”) 722, which can be a single signal, based on the linear combination of one or more of multiple color channel signals 702.

According to at least some examples, flushing signal 722 can be determined by “separating” the signal from multiple color channel signals 702 to form a time-domain component based on, for example, using Independent Component Analysis (“ICA”), such as a fitted ICA model, to estimate flushing signal 722 point-wise from red channel signal 703, green channel signal 704, and blue channel signal 705. An example of flushing signal 722 can be determined by equation (1) as follows:

ƒ(t)=wᵣr(t)+w_{g}g(t)+w_{b}b(t) Eqn. 1

whereby weights wᵣ, W_{g}, and w_{b} may correspond to a row, for example, in an ICA de-mixing matrix. In some cases, a fitted ICA can refer to Independent Component Analysis performed in view of, for example, the non-Gaussianity of source signals such that the original coordinate axes can be identified from an image. In a specific subset of examples, only the green color channel is used to determine flushing signal 722 by setting w_{g}=1 and setting wᵣ=wb=0 to reduce or negate effects of the iterative nature (e.g., batch computations) of ICA.

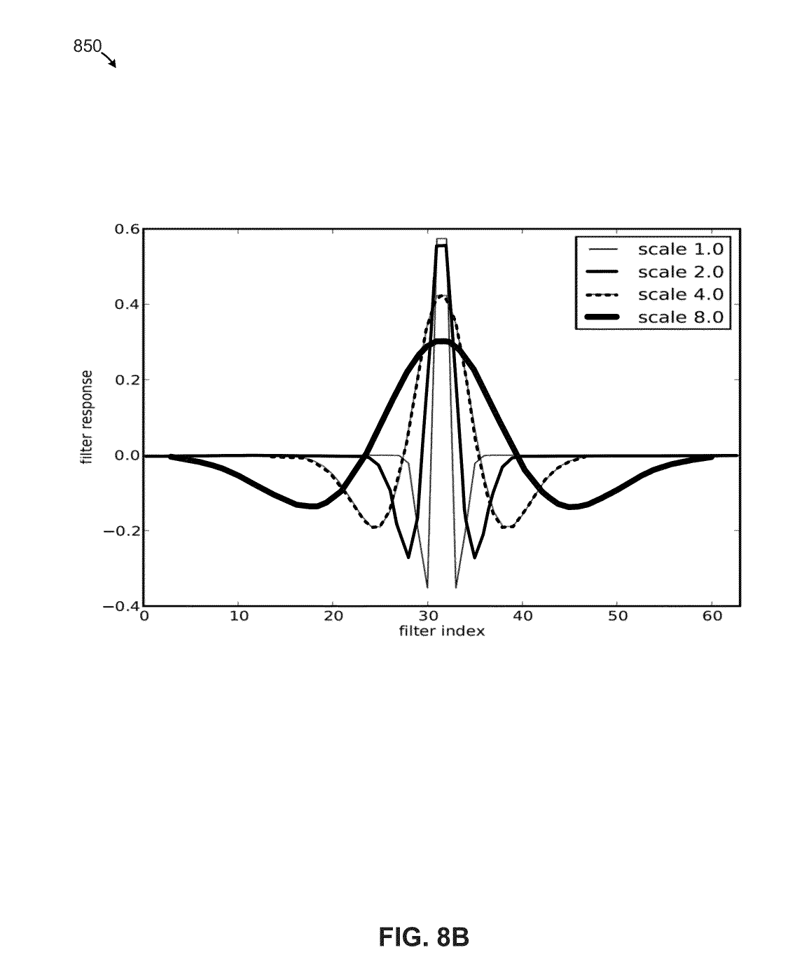

FIGS. 8A and 8B depict an example of a wavelet transformer, according to some embodiments. Diagram 800 of FIG. 8A depicts a wavelet transformer 820 that is configured to perform continuous wavelet transforms (“CWT”) in view of multiresolution analysis. As the flush pulses vary in width in the time dimension (e.g., durations of time of flushed states may vary in time). To reduce or negate effects of noise, wavelet transformer 820 can be configured to transform the combined linear color signal at multiple scales. Examples of threshold scales can range from two to several scales (or values of scale parameters, where such values can be integers or real numbers), or greater, in some cases.

An example of wavelet transformer 820 includes a filter bank 822, which include filters 823a, 823b, . . . , 823n, and a convolver 824. Filter bank 822 can be formed based on a wavelet shaped in continuous time, such as the “Mexican hat wavelet” or “Ricker wavelet,” which can be a normalized derivative of a Gaussian function parameterized by a scale parameter, a. Note that diagram 850 of FIG. 8B depicts several values of scale parameter a at, for example, 20 frames per second. Referring back to FIG. 8A, a wavelet transform can be expressed as follows:

$$ψ ( t ) = \frac{2}{\sqrt{3 σ} {π}^{\frac{1}{4}}} ( 1 - \frac{{t}^{2}}{{σ}^{2}} ) exp ( \frac{- {t}^{2}}{2 {σ}^{2}} ) , \qquad Eqn . 2$$

which is used to form filter bank 822 with filters 823a, 823b, . . . , 823n associated with different values of scale parameter, σ.

Convolver 824 is configured to apply filter bank 822 to a signal (e.g., a flushing signal, f(t), or a transformed combined linear color channel signal) by convolving filters 823a, 823b, . . . , 823n, with the combined linear signal (e.g., the transformed combined linear signal) at multiple scales, thereby measuring—at last conceptually—the similarity between the signal and the filters at each point in time. Further, convolver 824 is configured to map a continuous-time scalar signal function ƒ to a scalar response function according to:

$$F ( σ , t ) = \frac{1}{\sqrt{σ}} {\int }_{- ∞}^{+ ∞} f ( τ ) ψ ( \frac{τ - t}{σ} ) d τ , \qquad Eqn . 3$$

wherein the value of F (σ, t) can be relatively large when the signal ƒ at time t resembles locally a copy of the “Mexican hat wavelet” at scale σ. According to some examples, an inverse transform can be computed by integrating against a dual wavelet function, provided that the primal-dual wavelet pair can satisfy an inner product identity.

Plot 802 depicts results of a two-dimensional continuous wavelet transform of a green channel signal (e.g., a combined linear color channel signal) over multiple scales of the Mexican hat wavelet. Here, the value of scalar parameters can range from, for example, 1.0 to 3.9, or otherwise. The signal 808 is overlaid on plot 802 for comparison purposes only as the vertical positions and scaling selected for the plot is for illustration purposes. “Ridges” can be determined by the local maxima of the response functions should those local maxima exist at, or extend though, several contiguous scales. For example, local maximum 807 extends at least across scale values 3.9, 3.175, 2.45, and 1.725. In at least one view, the light shading remains consistent (e.g., a maximum value) for local maximum 807 and extends at least over all or most contiguous scale values 890 from 3.9 to 1.725, or less. Regardless, the uninterrupted intensity or shading for local maximum 807 indicates that it establishes a vertical “ridge,” with which to detect flushes (i.e., flushed states).

Note that a local minimum can be depicted with a dark shading, such as local minimum 805. Note, too, that the light shading of the local maxima, such as local maximum 807, can correspond to the “flush pulses,” which appears as “spikes,” in signal 808. Note further that a wavelet transform and the wavelets can be defined in terms of continuous-time functions for convenience as this facilitates simpler expressions and more flexible definitions. In particular, the definitions permit freedom to avoid the use of specific sample rates (or frame rates). When operating on a digital computer, the discrete-time signals generated by the computer can be viewed as sample points for the continuous-time signals, according to some examples.

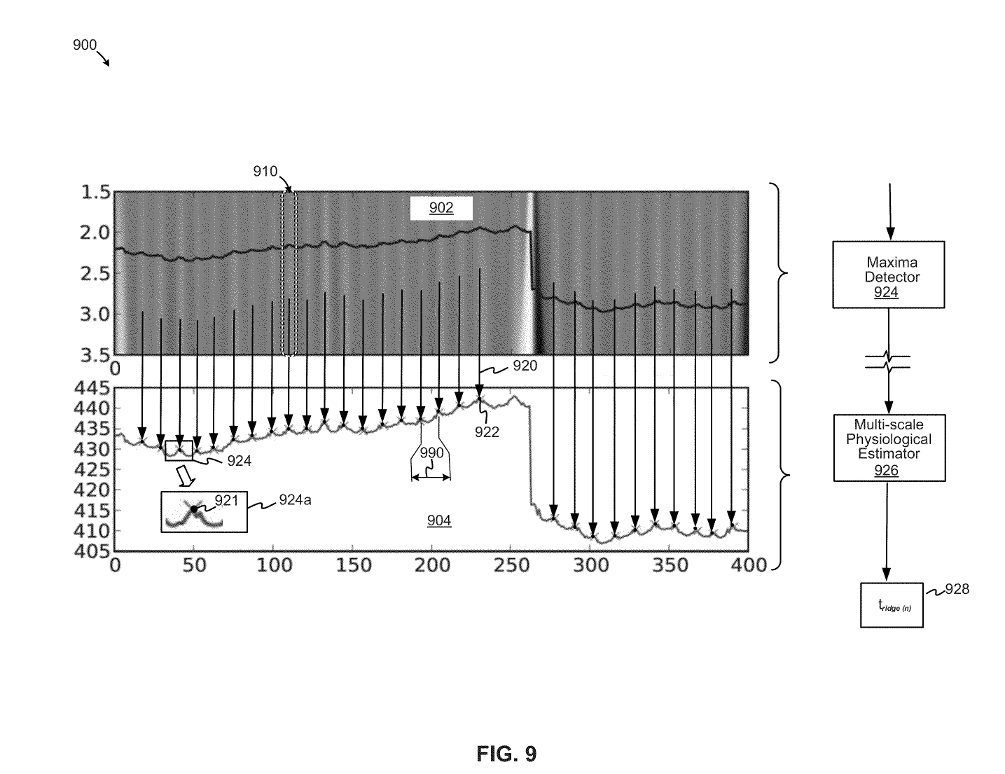

FIG. 9 depicts examples of a maxima detector and a multi-scale physiological estimator, according to some embodiments. Diagram 900 of FIG. 9 depicts a maxima detector 924 configured to detect local maxima (e.g., at light shaded vertical regions) in continuous wavelet transform 902, such as local maximum 910. Further, maxima detector 924 can determine that local maximum 910 and other local maxima establish “ridges,” which, in turn, corresponds to respective points 922 in time in signal plot 904. Arrows 920 extend from the local maxima depictions to identify points 922 in time. Multi-scale physiological estimator 926 is operable over multiple scales to identify, for example, timing 990 between points 922 in time. Thus, arrows 920 terminate at points 922, such a point 924, which is shown as enlarged spike 924a. As shown, point 921 is located at a spike (e.g., at a point in time associated with a flushing state). Multi-scale physiological estimator 926 then generates data representing the timing of ridges 928 with which to determine, for example, heart rate.

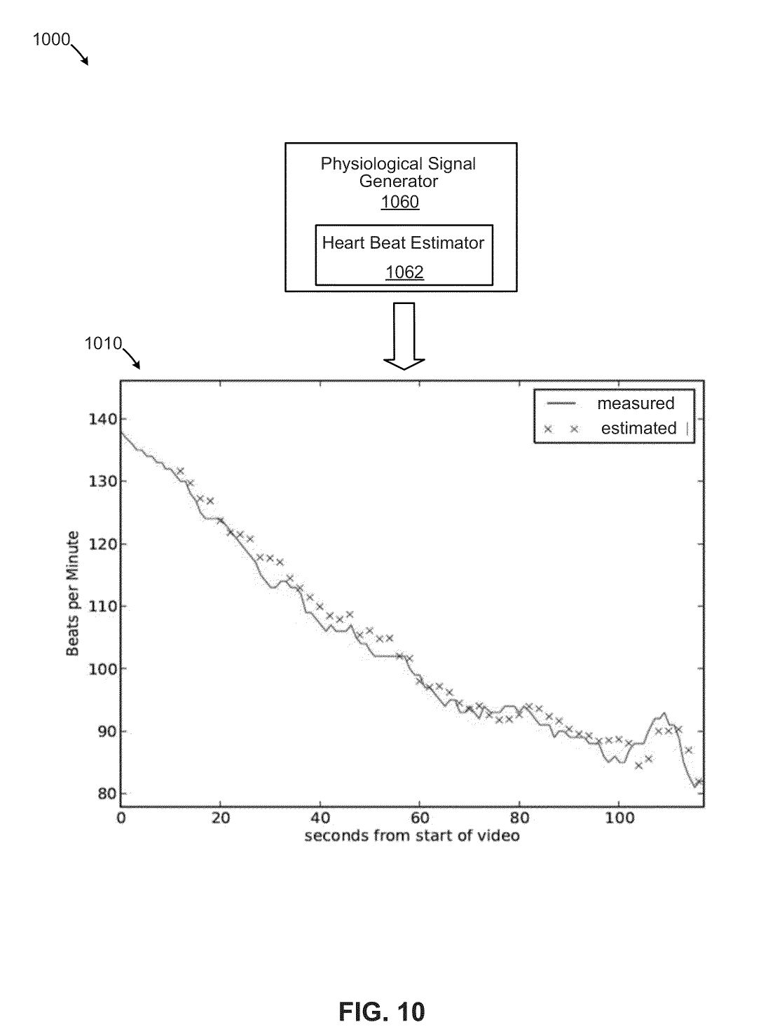

FIG. 10 depicts an example of a physiological signal generator, according to some embodiments. Diagram 1000 depicts physiological signal generator 1060 configured to generate data representing physiological characteristics, such as heart rate or heartbeat information. Further to diagram 1000, physiological signal generator 1060 includes a heartbeat estimator 1062, which is configured to receive timing information about ridges, for example, a multi-scale physiological estimator. Based on the timing information, heartbeat estimator 1062 can determine the number of heart beats per unit time, and, thereby estimate a heart rate. An example of heart rate estimation from a video signal is depicted in plot 1010, with estimates computed approximately every two seconds. Plot 1010 indicates a relatively good correlation between estimated heart rate and a measured heart rate, which is plotted as a control. Note that in some embodiments, a threshold, if met by the estimated heart rate, can cause performance of an action based on a value of the heart beats per unit time. Examples of an action is generating a display or a vibratory or electronic message to alert an interested party that a user is exceeding a number of heart beats per unit time, which perhaps may be dangerous or unhealthy.

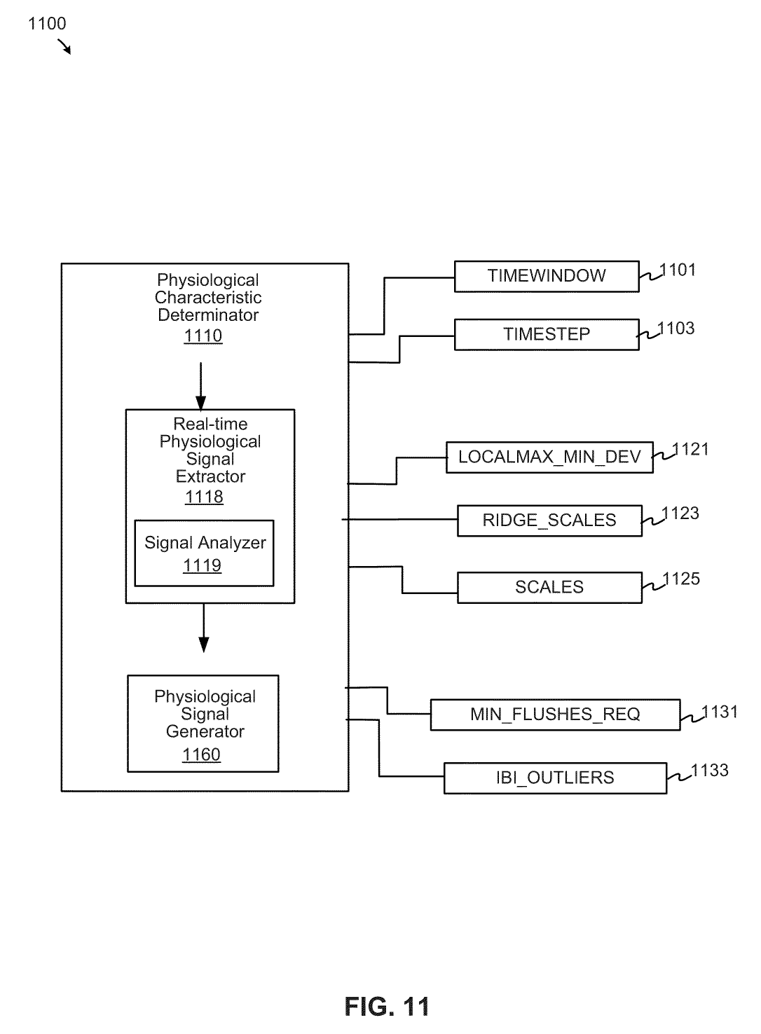

FIG. 11 depicts an example of a set of tunable parameters, according to some embodiments. Diagram 1100 depicts physiological characteristic determinator 1110 including a real-time physiological signal extractor 1118 and a physiological signal generator 1160. Also included is a signal analyzer 1119. One or more of these components can have structures and/or functions as similarly-named elements described elsewhere herein. According to some embodiments, one or more of the above-identified components can be implemented, for example, in Python programming language, as maintained by Python Software Foundation of Beaverton, Oreg., or variants (e.g., opens source scientific and numerical tools for Python maintained by SciPy Developers). In some examples, executable code for program scipy.signal.find_peaks_cwt can be used, with the following parameter data.

Also shown are parameter data 1101 to 1133 configured to tune operation of wavelet-based heart rate estimation. Parameter (“TIMEWINDOW”) data 1101 describes the number of seconds of the recent signal to use for estimating heart rate. The default value can be 12 seconds, but can range between 8 to 15 seconds. Parameter (“TIMESTEP”) data 1103 describes the number of seconds between new heart rate estimation of attempts. The default value can be 2 seconds.

Flush detection parameters include parameter data 1121, 1123, and 1125. Parameter (“LOCALMAX_MIN_DEV”) data 1121 describes a minimum deviation for a local maximum to be detected in a row of wavelet transforms (in standard units). The default value can be 0.5, as an example. Parameter (“RIDGE SCALES”) data 1123 describes a threshold proportion of contiguous scales at which local maxima in the wavelet transform for a ridge to be detected. The default value can be 0.75, as an example. Parameter (“SCALES”) data 1125 describes wavelet scales at which the wavelet transform is computed. This setting can depend on the data frame rate, which can be set at 20 frames per second as a default setting. The default value for SCALES can be 1.5:0.1:3.5, as an example.

Heart rate estimation parameters include parameter data 1131 and 1133. Parameter (“MIN_FLUSHES_REQUIRED”) data 1131 describes a minimum number of flushes detected within TIMEWINDOW to produce a heart rate estimate. The default value can be 5, but can range, for example, between 4 to 8. Parameter (“IBI_OUTLIERS”) data 1133 describes, within a window, inter-beat intervals further from the median than this number (in standard units) are considered outliers and can be ignored. The default value can be 1.5, but can range, for example, between 0.5 to 1.5.

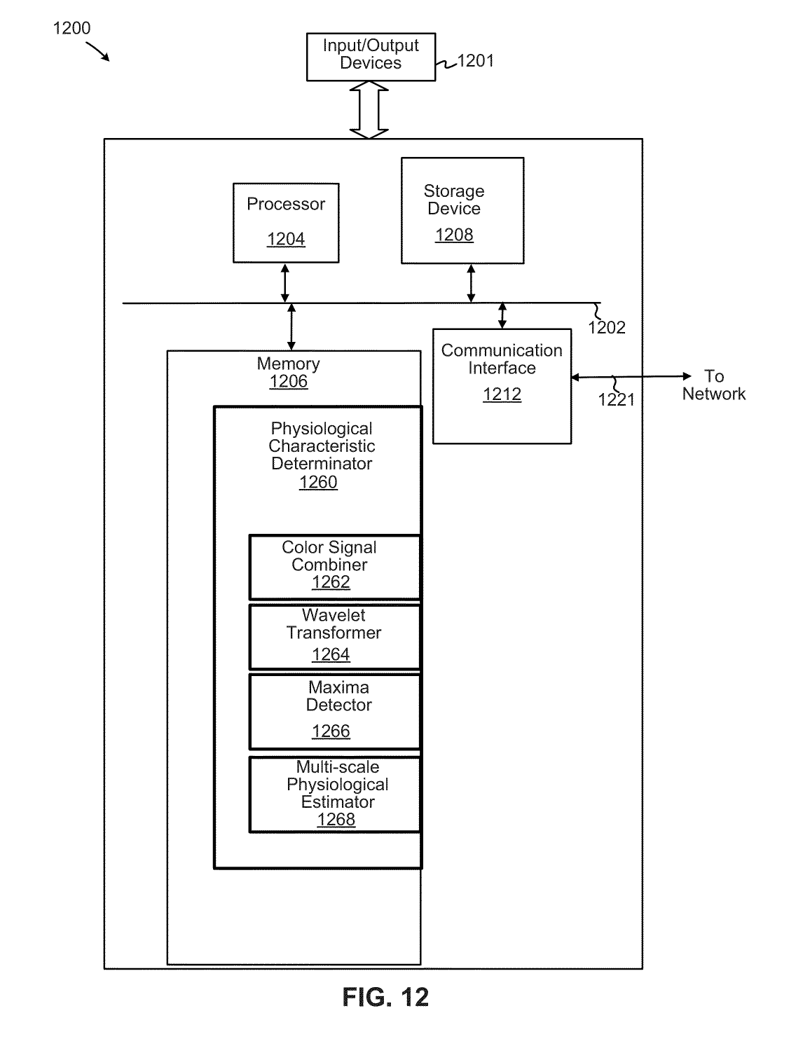

FIG. 12 illustrates an exemplary computing platform disposed in a media device, a mobile device, a wearable device, or any computing device, according to various embodiments. In some examples, computing platform 1200 may be used to implement computer programs, applications, methods, processes, algorithms, or other software to perform the above-described techniques. Computing platform 1200 includes a bus 1202 or other communication mechanism for communicating information, which interconnects subsystems and devices, such as processor 1204, system memory 1206 (e.g., RAM, etc.), storage device 1208 (e.g., ROM, etc.), a communication interface 1213 (e.g., an Ethernet or wireless controller, a Bluetooth controller, etc.) to facilitate communications via a port on communication link 1221 to communicate, for example, with a computing device, including mobile computing and/or communication devices with processors. Processor 1204 can be implemented with one or more central processing units (“CPUs”), such as those manufactured by Intel® Corporation, or one or more virtual processors, as well as any combination of CPUs and virtual processors. Computing platform 1200 exchanges data representing inputs and outputs via input-and-output devices 1201, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 1200 performs specific operations by processor 1204 executing one or more sequences of one or more instructions stored in system memory 1206, and computing platform 1200 can be implemented in a client-server arrangement, peer-to-peer arrangement, or as any mobile computing device, including smart phones and the like. Such instructions or data may be read into system memory 1206 from another computer readable medium, such as storage device 1208. In some examples, hard-wired circuitry may be used in place of or in combination with software instructions for implementation. Instructions may be embedded in software or firmware. The term “computer readable medium” refers to any tangible medium that participates in providing instructions to processor 1204 for execution. Such a medium may take many forms, including but not limited to, non-volatile media and volatile media. Non-volatile media includes, for example, optical or magnetic disks and the like. Volatile media includes dynamic memory, such as system memory 1206.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1202 for transmitting a computer data signal.

In some examples, execution of the sequences of instructions may be performed by computing platform 1200. According to some examples, computing platform 1200 can be coupled by communication link 1221 (e.g., a wired network, such as LAN, PSTN, or any wireless network) to any other processor to perform the sequence of instructions in coordination with (or asynchronous to) one another. Computing platform 1200 may transmit and receive messages, data, and instructions, including program code (e.g., application code) through communication link 1221 and communication interface 1213. Received program code may be executed by processor 1204 as it is received, and/or stored in memory 1206 or other non-volatile storage for later execution.

In the example shown, system memory 1206 can include various modules that include executable instructions to implement functionalities described herein. In the example shown, system memory 1206 (e.g., in a mobile computing device, or a wearable computing device) can include a physiological characteristic determinator module 1260 that includes a color signal combiner module 1262, a wavelet transformer module 1264, a Maxima detector module 1266, and a multi-scale physiological estimator module 1268.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

1. A method comprising:
receiving from an image capture device color channel signals including data representing one or more images;
forming a linear combination of the color channel signals as a combined color channel signal;
transforming continuously at a processor the combined color channel signal to establish local maxima associated with multiple scale parameters;
identifying portions of time associated with the local maxima;
deriving a physiological characteristic based on the portions of time; and
generating a data signal representing the physiological characteristic.

2. The method of claim 1, further comprising:
causing presentation at a display of the data representing the physiological characteristic.

3. The method of claim 1, wherein deriving a physiological signal comprises:
determining heart beats per unit time.

4. The method of claim 3, wherein deriving the physiological characteristic comprises:
determining the heart beats per unit time in real-time based on the combined color channel signal as a substantially continuous time signal.

5. The method of claim 3, further comprising:
causing performance of an action based on a value of the heart beats per unit time.

6. The method of claim 1, wherein receiving from the image capture device the color channel signals further comprises:
receiving light reflect from a surface of tissue,
wherein the portions of time associated with the local maxima indicate an enhanced blood volume state adjacent to the surface of the tissue.

7. The method of claim 1, wherein transforming continuously comprises:
performing a continuous wavelet transformation (“CWT”) of the combined color channel signal.

8. The method of claim 7, further comprising:
identifying a portions of time at which a local maximum exists at substantially adjacent values of scale parameters; and
determining the local maximum over the substantially adjacent values of scale parameters constitute a ridge.

9. The method of claim 7, wherein performing the continuous wavelet transformation comprises:
applying the continuous wavelet transform over the multiple scale parameters;
identifying a local maximum over values of a contiguous subset of the multiple scale parameters at a portion of time; and
determining a ridge,
wherein the ridge indicates presence of a flush state for a portion of tissue.

10. The method of claim 9, further comprising:
identifying multiple ridges over multiple portions of time;
determine timing between multiple flush states over a subset of the multiple portions of time; and
estimating a heart rate based on the timing.

11. The method of claim 7, wherein performing the continuous wavelet transformation comprises:
applying a Ricker wavelet to the combined color channel signal.

12. The method of claim 1, wherein transforming continuously comprises:
identifying a subset of scale parameters; and
convolving at the processor the combined color channel signal with a continuous wavelet transformation at each value of a scale parameter from the subset of scale parameters.

13. The method of claim 1, wherein receiving the color channel signals further comprises:
receiving a signal including a red color channel, a green color channel, and a blue color channel as the color channel signals.

14. The method of claim 2, wherein forming the linear combination further comprises:
forming the combined color channel based substantially on the green color channel.

15. An apparatus comprising:
a processor configured to execute instructions; and
a memory including modules of executable instructions, the modules comprising:
executable instructions to implement a continuous wavelet transformer module configured to transform continuously a combined color channel signal to establish local maxima associated with multiple scale parameters;
executable instructions to implement a maxima detector module configured to identifying portions of time associated with the local maxima for the combined color channel signal, the local maxima specifying a range of frequencies of light associated with a presence of an amount of blood adjacent a tissue surface from which the light in the combined color channel signal originates; and
executable instructions to implement a multi-scale physiological estimator configured to estimate heart beats per unit time in real-time based on the rate at which the portions of time occur over at least a subset of the multiple scale parameters.

16. The apparatus of claim 15, wherein the memory further comprises:
executable instructions to implement a color signal combiner module configured to generate a linear combination of a red color channel, a green color channel, and a blue color channel to form the combined color channel signal.

17. The apparatus of claim 16, further comprises:
an image capture configured to generate the red color channel, the green color channel, and the blue color channel based on the light reflected from the tissue surface.

18. The apparatus of claim 16, further comprises:
a housing in which the processor and the memory are disposed, the housing configured to couple or attach to an appendage.

19. The apparatus of claim 18, wherein the apparatus is a wearable computing device configured to be worn at a wrist.
